(19) United States
(12) Patent Application Publication
(10) Pub. No.: US 2025/0367262 A1
(43) Pub. Date: Dec. 4, 2025

(54) COMPOSITIONS AND METHODS FOR INTRAUTERINE INSEMINATION (IUI)

(71) Applicant: FERRING B.V., Hoofddorp (NL)

(72) Inventors: Francois BISSONNETTE, Montreal (CA); Isaac-Jacques KADOCH, Montreal (CA)

(73) Assignee: FERRING B.V., Hoofddorp (NL)

(21) Appl. No.: 18/874,466

(22) PCT Filed: Jul. 10, 2023
(86) PCT No.: PCT/EP2023/068982

§ 371 Date: Dec. 12, 2024

(30) Foreign Application Priority Data
Jul. 8, 2022 (EP) 22183901.2

(51) Int. Cl. A61K 38/24; A61B 17/43; A61P 15/08

(52) U.S. Cl. CPC A61K 38/24; A61B 17/43; A61P 15/08

(57) ABSTRACT

Methods, uses and compositions including FSH for use in the treatment of infertility by intrauterine insemination are described.

The present invention relates to methods, compositions and pharmaceutical products for the treatment of infertility.

### BACKGROUND

Assisted reproductive technologies (ART) such as in vitro fertilisation (IVF) are well known. ART generally requires a step of controlled ovarian stimulation (COS), in which a cohort of follicles is stimulated to full maturity. Standard COS regimens include administration of gonadotrophins, such as follicle stimulating hormone (FSH), alone or in combination with luteinising hormone (LH) activity to stimulate multiple follicular development. Usually COS requires administration of a GnRH analogue (GnRH agonist) or GnRH antagonist prior to and/or during stimulation to prevent a premature LH surge which may induce ovulation before planned oocyte retrieval. The aim of COS is generally to stimulate a number of follicles to maturity so, when ovulation is triggered, a number of oocytes are retrieved for fertilization. In general, the best is selected for implantation while others may be cryopreserved for future use.

In case of a too high ovarian response, COS can be associated with a risk of ovarian hyperstimulation syndrome (OHSS), which can lead to cancellation of the COS cycle and can become life threatening in severe cases. The ability to predict the ovarian response potential of women to COS may allow the development of personalised or individualised COS protocols. Such individualised protocols could, for example, reduce the risk of OHSS in women predicted to have an excessive ovarian response to COS, and prevent cancellation of COS cycles. Levels of Anti Mullerian Hormone (AMH) are directly correlated with the ovarian response to gonadotrophins during COS. Thus, high levels of AMH are a good predictor of excessive ovarian response, and an indicator of risk of OHSS, whereas low levels of AMH predict a poor ovarian response to COS.

Clinical research has focused the last years on the development of individualised dosing regimens for COS, initially without using AMH but based on other predictors of ovarian response. These predictors include age, body mass index (BMI), FSH, and antral follicle count (AFC).

As indicated above, standard COS protocols require daily FSH administration to induce multiple follicular growth to obtain sufficient oocytes for IVF. FSH is a natural hormone that is secreted by the anterior pituitary gland. In healthy women FSH induces monthly the growth of a single dominant follicle that ovulates during each natural cycle. FSH purified from the urine of post-menopausal women has been used for many years in infertility treatment, both to promote ovulation in natural reproduction and to induce multiple follicular growth to obtain sufficient oocytes for ART.

Until recently, the only approved recombinant FSH (rFSH) products for ovarian stimulation, such as follitropin alfa (GONAL-F®, Merck Serono/EMD Serono) and follitropin beta (PUREGON®/FOLLISTIM®, MSD/Schering-Plough), were derived from a Chinese Hamster Ovary (CHO) cell line and have only 2,3-sialylation. The present applicants have developed a human cell line-derived rFSH which is the subject of International Patent Application No. PCT/GB2009/000978, published as WO2009/127826A. On 13 Dec. 2016, the European Commission (EC) granted marketing authorisation for REKOVELLE® (follitropin delta, also known as FE 999049), a human cell line-derived recombinant follicle stimulating hormone (human rFSH), for use in controlled ovarian stimulation for the development of multiple follicles in women undergoing assisted reproductive technologies (ART), such as an in vitro fertilisation (IVF) cycle. REKOVELLE® is the first rFSH to be derived from a human cell line. The REKOVELLE® (follitropin delta) product is produced by the methods disclosed in International Patent Application No. PCT/GB2009/000978.

In the context of IVF, the posology of REKOVELLE® is individualised for each patient and aims to obtain an ovarian response which is associated with a favourable safety/efficacy profile, i.e. aims to achieve an adequate number of oocytes retrieved and reduce the interventions needed to prevent OHSS. REKOVELLE® is dosed in micrograms (ug). The individualised dose may be modelled to achieve 8-14 oocytes in ART cycles based on AMH and body weight. To this end, for the first treatment cycle, an individual daily dose may be determined on the basis of the woman's serum AMH concentration (also referred to herein as “serum AMH level”) and, depending on serum AMH concentration, her body weight. The dose may be based on a recent determination of AMH (i.e., within the last 24 months, or within the last 12 months) measured by the ELECSYS® AMH Plus immunoassay (Roche). The individual daily dose may be maintained throughout the stimulation period. For women with AMH <15 pmol/L the daily dose of REKOVELLE® may be 12 ug, irrespective of body weight. For women with AMH ≥15 pmol/L the daily dose of REKOVELLE® may be lower, and may range from 0.19 ug/kg to 0.10 ug/kg over AMH concentrations of 15 to ≥40 pmol/L, where the maximum dose in the first cycle is 12 ug. For subsequent treatment cycles, the daily dose of REKOVELLE® may be maintained or may be modified according to the patient's ovarian response in the previous cycle. If the patient had adequate ovarian response in the previous cycle without developing OHSS, the same daily dose may be used. In case of ovarian hypo-response in the previous cycle, the daily dose in the subsequent cycle may be increased by 25% or 50%, according to the extent of response observed. In case of ovarian hyper-response in the previous cycle, the daily dose in the subsequent cycle may be decreased by 20% or 33%, according to the extent of response observed. In patients who developed OHSS or were at risk of OHSS in a previous cycle, the daily dose for the subsequent cycle may be 33% lower than the dose the cycle where OHSS or risk of OHSS occurred. The maximum daily dose of REKOVELLE® typically is 24 ug.

Intrauterine insemination (IUI) is a fertility treatment where sperm is placed directly into the uterus using a small catheter. It may be used, for example, in situations where vaginal sexual intercourse is not possible (for example because of physical disability or psychosexual problem), or to treat couples experiencing infertility due to medical conditions (e.g., endometriosis or low sperm count or quality), or couples with unexplained infertility. IUI is widely used because it is a minimally invasive, lower-cost alternative to in vitro fertilization (IVF), and it can be conveniently performed in a clinic. The goal of IUI is to improve the chances of fertilization by increasing the number of healthy sperm that reach the fallopian tubes when the woman is most fertile. IUI may be performed on patients who undergo ovarian stimulation to increase the likelihood of a positive outcome. Unlike COS used in IVF and ART, however, where the aim is to obtain 8-14 oocytes prior to triggering ovulation, the aim for IUI is to obtain only two to three follicles ≥16 mm on the day prior to or on the day of triggering ovulation, and to avoid multiple pregnancies.

There is a need for individualised stimulation protocols which provide improved pregnancy outcomes in patients undergoing IUI.

### SUMMARY

In the proof-of-concept study described below, the applicants demonstrated the effectiveness of a personalised dose regimen for REKOVELLE® in IUI based on AMH and bodyweight. The percentage of patients treated who obtained 2 or 3 mature follicles (defined as a follicle >16 mm on the day prior to or on the day of triggering ovulation and/or, e.g., as detected in the last ultrasound scan before triggering) in their first, second and third cycles was 34%, 37% and 47% respectively. The percentage of patients who reached more than 3 follicles was 1.9%, 0% and 1.5% respectively. Thus, the dosing regimen described herein achieves the desired goal. Only 6 multiple pregnancies out of 40 clinical pregnancies (15%) were observed (twins only), showing the dosing regimen is a safe approach. The clinical pregnancy rate per IUI cycle by intention to treat was 16%, 14.3% and 16.2% for the 3 IUI cycles, respectively. The cumulative clinical pregnancy rate over the 3 cycles among patients completing the study was 37.7%, higher than previous studies using conventional dosing of rFSH which has only 2,3-sialylation, indicative of usefulness for clinical practice.

In accordance with some aspects, there are provided compositions comprising follicle stimulating hormone (FSH) for use in the treatment of infertility by intrauterine insemination (e.g. treatment of infertility by controlled ovarian stimulation for intrauterine insemination) [e.g. to increase pregnancy rate and/or live birth rate, e.g. to increase pregnancy rate and/or live birth rate compared to treatment with rFSH which includes only 2,3-sialylation, e.g. to increase pregnancy rate and/or live birth rate compared to fixed dose treatment with rFSH which includes only 2,3-sialylation] in a patient having a serum AMH level of <15 pmol/L, wherein the composition is to be administered at a dose of, or equivalent to, 0.048 ug to 0.052 ug, for example 0.05 ug, recombinant FSH per kg bodyweight of the patient per day. The use may comprise a step of determining the serum AMH level of the patient, and a step of administering the dose to a patient having serum AMH level of <15 pmol/L. In some aspects, the composition is to be administered at a dose of 0.05 ug recombinant FSH per kg bodyweight of the patient per day. In some aspects the FSH is recombinant FSH, for example recombinant FSH which includes α2,3- and α2,6-sialylation, for example recombinant FSH which includes α2,3- and α2,6-sialylation wherein 5% to 20% of the total sialylation is α2,6-sialylation and wherein 80% to 95% of the total sialylation is α2,3-sialylation. In some aspects, the FSH is a recombinant FSH which has been produced or expressed in a human cell line. The use may comprise a further step of triggering ovulation (by e.g. administering a dose of or dose equivalent to 4,000 to 11,000 IU hCG). The use may comprise a further step of intrauterine insemination. The treatment of infertility by intrauterine insemination may be controlled ovarian stimulation for intrauterine insemination [e.g. controlled ovarian stimulation to prepare the patient for (a subsequent step of) intrauterine insemination]. The treatment of infertility by intrauterine insemination may include controlled ovarian stimulation (e.g. using rFSH having α2,3- and α2,6-sialylation) with the aim of obtaining 2 or 3 mature follicles (defined as a follicle ≥16 mm on the day before or on the day of triggering ovulation and/or, e.g., as detected in the last ultrasound scan before triggering) for a subsequent step of intrauterine insemination.

In accordance with some aspects, there are provided compositions comprising follicle stimulating hormone (FSH) for use in the treatment of infertility by intrauterine insemination (e.g. treatment of infertility by controlled ovarian stimulation for intrauterine insemination) [e.g. to increase pregnancy rate and/or live birth rate, e.g. to increase pregnancy rate and/or live birth rate compared to treatment with rFSH which includes only 2,3-sialylation, e.g. to increase pregnancy rate and/or live birth rate compared to fixed dose treatment with rFSH which includes only 2,3-sialylation] in a patient having serum AMH level of ≥15 pmol/L (for example serum AMH of ≥23 pmol/L), wherein the composition is to be administered at a dose of, or equivalent to, 0.025 to 0.0475 ug recombinant FSH per kg bodyweight of the patient per day. In some aspects the patient may be a patient with polycystic ovary syndrome (PCOS). In some aspects the patient may be a patient with PCOS with serum AMH of ≥23 pmol/L. In accordance with some aspects, there are provided compositions comprising follicle stimulating hormone (FSH) for use in the treatment of infertility by intrauterine insemination (e.g. treatment of infertility by controlled ovarian stimulation for intrauterine insemination) [e.g. to increase pregnancy rate and/or live birth rate, e.g. to increase pregnancy rate and/or live birth rate compared to treatment with rFSH which includes only 2,3-sialylation e.g. to increase pregnancy rate and/or live birth rate compared to fixed dose treatment with rFSH which includes only 2,3-sialylation] in a patient with PCOS having a serum AMH level of ≥15 pmol/L, wherein the composition is to be administered at a dose of, or equivalent to, 0.025 to 0.0475 μg recombinant FSH per kg bodyweight of the patient per day. In some aspects the composition is to be administered at a dose of 0.025 to 0.0475 μg recombinant FSH per kg bodyweight of the patient per day. In some aspects the FSH is recombinant FSH, for example recombinant FSH which includes α2,3- and α2,6-sialylation, for example recombinant FSH which includes α2,3- and α2,6-sialylation wherein 5% to 20% of the total sialylation is α2,6-sialylation and wherein 80% to 95% of the total sialylation is α2,3-sialylation. In some aspects, the FSH is a recombinant FSH which has been produced or expressed in a human cell line. The use may comprise a step of determining the serum AMH level of the patient, and a step of administering the dose to a patient having serum AMH level of ≥15 pmol/L. The use may comprise a further step of triggering ovulation (by e.g. administering a dose of or dose equivalent to 4,000 to 11,000 IU hCG). The use may comprise a further step of intrauterine insemination. The treatment of infertility by intrauterine insemination may be controlled ovarian stimulation for intrauterine insemination [e.g. controlled ovarian stimulation to prepare the patient for (a subsequent step of) intrauterine insemination]. The treatment of infertility by intrauterine insemination may include controlled ovarian stimulation (e.g. using rFSH having α2,3- and α2,6-sialylation) with the aim of obtaining 2 or 3 mature follicles (defined as a follicle ≥16 mm on the day before or on the day of triggering ovulation, and/or, e.g., as detected in the last ultrasound scan before triggering) for a subsequent step of intrauterine insemination.

The compositions may be for administration at the defined dose of rFSH (e.g. that is, the starting dose of rFSH) from day 1 to day 6 of treatment, that is from day 1 to day 6 of stimulation. From day 7 of treatment (day 7 of stimulation) the dose of rFSH may be: increased (e.g. increased to a dose of up to 150% of the starting dose, for example increased to a dose of 120% to 150% of the starting dose); decreased (e.g. decreased to a dose of 50% of the starting dose, for example decreased to a dose of 50% to 80% of the starting dose); or maintained at the starting dose. The dose may be increased, decreased or maintained based on the patient's ovarian response (e.g. as measured by ultrasonography).

The (e.g. starting) doses disclosed herein may be the doses for a first cycle of IUI. For second and subsequent cycles of IUI the “starting dose” may be: increased (e.g. increased to a dose of up to 150% of the starting dose for the first cycle, for example increased to a dose of 120% to 150% of the starting dose for the first cycle); decreased (e.g. decreased to a dose of 50% of the starting dose for the first cycle, for example decreased to a dose of 50% to 80% of the starting dose for the first cycle); or maintained at the staring dose for the first cycle. The dose may be increased, decreased or maintained based on the patient's response to the first cycle of IUI (e.g. as measured by number of mature follicles ≥16 mm obtained in the first cycle on the day prior to or on the day of triggering, and/or, e.g., as detected in the last ultrasound scan before triggering).

In accordance with some aspects, there is provided a method of treatment of infertility by intrauterine insemination (e.g., a method of treatment of infertility by controlled ovarian stimulation for intrauterine insemination) in a patient having a serum AMH level of <15 pmol/L, the method comprising administering to the patient a dose of, or equivalent to, from 0.048 μg to 0.052 μg recombinant FSH (rFSH) per kg bodyweight of the patient per day, for example, 0.05 μg rFSH per kg bodyweight of the patient per day, optionally wherein the rFSH includes α2,3- and α2,6-sialylation. The method may be a method of increasing one or both of pregnancy rate and live birth rate, e.g., a method of increasing one or both of pregnancy rate and live birth rate compared to treatment with rFSH which includes only 2,3-sialylation, e.g., a method of increasing one or both of pregnancy rate and live birth rate compared to fixed dose treatment with rFSH which includes only 2,3-sialylation. The method may comprise, prior to the administering, determining the serum AMH level of the patient, and administering the rFSH dose to a patient having serum AMH level of <15 pmol/L. In some aspects, the rFSH is administered at a dose of 0.05 μg rFSH per kg bodyweight of the patient per day. In some aspects, the rFSH includes α2,3- and α2,6-sialylation, for example rFSH which includes α2,3- and α2,6-sialylation wherein 5% to 20% of the total sialylation is α2,6-sialylation and wherein 80% to 95% of the total sialylation is α2,3-sialylation. In some aspects, the rFSH has been produced or expressed in a human cell line, such as the PER.C6® cell line. The method may further comprise triggering ovulation (e.g., by administering hCG at a dose of 4,000 to 11,000 IU hCG). The method may further comprise a step of intrauterine insemination. The treatment of infertility by intrauterine insemination may be controlled ovarian stimulation for intrauterine insemination, e.g., controlled ovarian stimulation to prepare the patient for (a subsequent step of) intrauterine insemination. The treatment of infertility by intrauterine insemination may include controlled ovarian stimulation (e.g. using rFSH having α2,3- and α2,6-sialylation) with the aim of obtaining 2 or 3 mature follicles (where “mature follicle” is defined as a follicle ≥16 mm on the day before or on the day of triggering ovulation, and/or, e.g., as detected in the last ultrasound scan before triggering) for a subsequent step of intrauterine insemination.

In accordance with some aspects, there is provided a method of treatment of infertility by intrauterine insemination (e.g., a method of treatment of infertility by controlled ovarian stimulation for intrauterine insemination) in a patient having a serum AMH level of ≥15 pmol/L (for example serum AMH of ≥23 pmol/L), the method comprising administering to the patient a dose of, or equivalent to, from 0.025 to 0.0475 μg recombinant FSH (rFSH) per kg bodyweight of the patient per day, optionally wherein the rFSH includes α2,3- and α2,6-sialylation. In some aspects, the patient may be a patient with polycystic ovary syndrome (PCOS). The method may be a method of increasing one or both of pregnancy rate and live birth rate, e.g., a method of increasing one or both of pregnancy rate and live birth rate compared to treatment with rFSH which includes only 2,3-sialylation, e.g., a method of increasing one or both of pregnancy rate and live birth rate compared to fixed dose treatment with rFSH which includes only 2,3-sialylation. In some aspects the rFSH is administered at a dose of from 0.025 to 0.0475 μg rFSH per kg bodyweight of the patient per day based on the patient's serum AMH level, as indicated below:

| Measured AMH | Rounded AMH | AMH | Daily Dose | |
|---|---|---|---|---|
| pmol/L | pmol/L | ng/mL | of rFSH | |
| 14.5-16.4 | 15-16 | 2.1-2.24 | 0.0475 | μg/kg |
| 16.5-17.4 | 17 | 2.38 | 0.045 | μg/kg |
| 17.5-18.4 | 18 | 2.52 | 0.0425 | μg/kg |
| 18.5-20.4 | 19-20 | 2.66-2.8 | 0.04 | μg/kg |
| 20.5-22.4 | 21-22 | 2.94-3.08 | 0.0375 | μg/kg |
| 22.5-24.4 | 23-24 | 3.22-3.36 | 0.035 | μg/kg |
| 24.5-27.4 | 25-27 | 3.5-3.78 | 0.0325 | μg/kg |
| 27.5-32.4 | 28-32 | 3.92-4.48 | 0.03 | μg/kg |
| 32.5-39.4 | 33-39 | 4.62-5.46 | 0.0275 | μg/kg |
| >39.5 | >40 | >5.6 | 0.025 | μg/kg |

In some aspects the rFSH includes α2,3- and α2,6-sialylation, for example, rFSH which includes α2,3- and α2,6-sialylation wherein 5% to 20% of the total sialylation is α2,6-sialylation and wherein 80% to 95% of the total sialylation is α2,3-sialylation. In some aspects, the rFSH has been produced or expressed in a human cell line, such as the PER.C6@ cell line. The method may comprise, prior to the administering, determining the serum AMH level of the patient, and administering the rFSH dose to a patient having serum AMH level of ≥15 pmol/L. The method may further comprise triggering ovulation (e.g., by administering hCG at a dose of 4,000 to 11,000 IU hCG). The method may further comprise a step of intrauterine insemination. The treatment of infertility by intrauterine insemination may be controlled ovarian stimulation for intrauterine insemination, e.g., controlled ovarian stimulation to prepare the patient for (a subsequent step of) intrauterine insemination. The treatment of infertility by intrauterine insemination may include controlled ovarian stimulation (e.g., using rFSH having α2,3- and α2,6-sialylation) with the aim of obtaining 2 or 3 mature follicles (where “mature follicle” is defined as a follicle ≥16 mm on the day before or on the day of triggering ovulation, and/or, e.g., as detected in the last ultrasound scan before triggering) for a subsequent step of intrauterine insemination.

### DETAILED DESCRIPTION

The present applicants have demonstrated the effectiveness of a personalised dose regimen for rFSH including α2,3- and α2,6-sialylation (e.g., wherein 5% to 20% of the total sialylation is α2,6-sialylation and wherein 80% to 95% of the total sialylation is α2,3-sialylation) in IUI (e.g., treatment of infertility by controlled ovarian stimulation for intrauterine insemination) based on AMH and bodyweight. The percentage of patients treated who obtained 2 or 3 mature follicles (with “mature follicle” defined as a follicle ≥16 mm on the day before or on the day of triggering ovulation, and/or, e.g., as detected in the last ultrasound scan before triggering) in their first, second and third cycles of IUI was 34%, 37% and 47%, respectively. The percentage of patients who reached more than 3 follicles was 1.9%, 0% and 1.5%, respectively. Thus, the dosing regimen described herein achieves the desired goal. Only 6 multiple pregnancies out of 40 clinical pregnancies (15%) were observed, showing this dosing regimen is a safe approach. The clinical pregnancy rate per IUI cycle by intention to treat was 16%, 14.3% and 16.2% for the 3 IUI cycles, respectively. The cumulative clinical pregnancy rate over the 3 cycles among patients completing the study was 37.7%, indicative of usefulness for clinical practice.

Therefore, provided herein are treatments and dosing regimens constructed for use to treat a patient for infertility by IUI.

### Definitions

Technical and scientific terms used herein have the meanings commonly understood by one of ordinary skill in the art of assisted reproductive technology to which the present invention pertains, unless otherwise defined. Reference is made herein to various methodologies known to those of ordinary skill in the art. Any suitable materials and/or methods known to those of ordinary skill in the art can be utilized in carrying out the present invention based on the guidance provided herein. However, specific materials and methods are described. Materials, reagents and the like to which reference is made in the following description and examples are obtainable from commercial sources, unless otherwise noted.

It is to be understood, that any definitions and terms herein defined is meant to have the same meaning and purpose in any of the aspects and embodiments of the invention unless explicitly otherwise stated not to.

As used herein, the singular forms “a,” “an,” and “the” designate both the singular and the plural, unless expressly stated to designate the singular only.

As used herein, the term “about” means that the number or range is not limited to the exact number or range set forth, but encompass ranges around the recited number or range as will be understood by persons of ordinary skill in the art depending on the context in which the number or range is used. Unless otherwise apparent from the context or convention in the art, “about” mean up to plus or minus 10% of the particular term.

Herein the terms “patient” and “subject” and “female” and “woman” are used interchangeably.

Herein the term “treatment of infertility” includes treatment of infertility by controlled ovarian stimulation (COS) or methods which include a step or stage of controlled ovarian stimulation (COS). The term “treatment of infertility” includes treatment of infertility in a subject having tubal or unexplained infertility, including treatment of infertility in a subject having endometriosis, for example stage I or stage II endometriosis, and/or in a subject with a partner with male factor infertility. The compositions and methods described herein may be for (use in) the treatment of infertility (and/or for controlled ovarian stimulation) in a subject having endometriosis, for example in a subject having stage I or stage II endometriosis, as defined by The American Society for Reproductive Medicine (ASRM) classification system for the various stages of endometriosis, (stage IV most severe; stage I least severe). See American Society for Reproductive Medicine. Revised American Society for Reproductive Medicine Classification of Endometriosis: 1996. Fertil. Steril. 1997; 67,817 821.

The term “treatment of infertility by intrauterine insemination” as used herein includes controlled ovarian stimulation for intrauterine insemination. In other words, the treatment of infertility by intrauterine insemination includes controlled ovarian stimulation (using, e.g., rFSH having α2,3- and α2,6-sialylation) with the aim of obtaining 2 or 3 mature follicles (with “mature follicle” defined as a follicle ≥16 mm on the day before or on the day of triggering ovulation, and/or, e.g., as detected in the last ultrasound scan before triggering) for a subsequent step of intrauterine insemination.

The term “positive pregnancy test” as used herein is defined as a positive urine pregnancy test. The term “clinical pregnancy” as used herein is defined as a viable intrauterine pregnancy at 6-8 weeks confirmed by an ultrasound scan. The term “ongoing pregnancy” as used herein is defined as a viable intrauterine pregnancy of at least 12 weeks duration confirmed on an ultrasound scan. The term “pregnancy loss” as used herein is defined as the spontaneous demise of a pregnancy before 24 weeks of gestational age. The term “multiple pregnancy rate” as used herein is defined as visualisation of two or more embryos with a fetal heartbeat at the viability ultrasound between 6 and 8 weeks of pregnancy per total number of clinical pregnancies.

As used herein, the term “oligoovulation” refers to infrequent or irregular ovulation amounting to eight (8) or fewer menstrual cycles (periods) per year, and may include women with cycles of >31 days. As used herein, the phrases a patient “identified with oligoovulation” or “diagnosed with oligoovulation” and a patient “with oligoovulation,” are used interchangeably to refer to a patient who has 8 or fewer menstrual cycles (periods) in a year, and excludes anovulatory patients. Oligoovulation is one of the most common causes of infertility for women.

As used herein, the term “anovulatory” or “anovulation” refers to a patient whose ovaries do not release an oocyte during a menstrual cycle. Therefore, ovulation does not take place. Chronic anovulation is a common cause of infertility.

Polycystic ovary syndrome (PCOS) is one of the most common endocrine disorders affecting women of reproductive age. The clinical features may include hyperandrogenism (with the clinical manifestations of oligomenorrhoea, hirsutism, and acne), ovulation disorders (such as infrequent, irregular and/or prolonged menstrual cycles), and polycystic ovarian morphology. In the UK, the National Institute for Health and Care Excellence (NICE) Website states that in adults, PCOS should be diagnosed if two out of three of the following criteria are present, provided other causes of menstrual disturbance and hyperandrogenism have been excluded: Clinical and/or biochemical signs of hyperandrogenism; Oligo-anovulation or anovulation; and Polycystic ovaries, defined as the presence of 12 or more follicles (measuring 2-9 mm in diameter) in one or both ovaries and/or increased ovarian volume (more than 10 cm³).

As used here “Patient with Polycystic Ovarian Syndrome” or “Patient with PCOS” refers to a patient (woman) who has PCOS, including a patient who has been diagnosed as having PCOS. The patient with PCOS may have two or more of the following criteria (typically for diagnosis of PCOS other causes of menstrual disturbance and hyperandrogenism have been excluded): Clinical and/or biochemical signs of hyperandrogenism; oligo-anovulation or anovulation; and polycystic ovaries, defined as the presence of 12 or more follicles (measuring 2-9 mm in diameter) in one or both ovaries and/or increased ovarian volume (more than 10 cm³). To date (June 2023), PCOS guidelines have indicated that serum AMH levels should not be used as an alternative for the detection of polycystic ovarian morphology (PCOM) or as a single test for the diagnosis of PCOS. However, there is emerging evidence that with improved standardisation of assays and established cut off levels or thresholds based on large scale validation in populations of different ages and ethnicities, AMH assays will be more accurate in the detection of PCOM and as a single test for the diagnosis of PCOS. Herein, the term “Patient with Polycystic Ovarian Syndrome” or “Patient with PCOS” may additionally refer to a patient (woman) who has PCOS, including a patient who has been diagnosed as having PCOS, who has serum AMH of 23 pmol/L or more (based on new ESHRE guidelines).

The term “follicle” herein means an ovarian follicle which is a fluid-filled sac that contains an immature egg, or oocyte.

A blastocyst forms in the early development of a human (or other mammal). In humans, blastocyst formation begins about 5 days after fertilization. The use of blastocysts in IVF generally involves retrieval (harvesting) from the woman a number of oocytes resulting from a controlled ovarian stimulation cycle; fertilization (insemination of) one or more oocytes and culturing the fertilized egg (oocyte) for five days to form a blastocyst (i.e. allowing the fertilized oocyte to develop to the blastocyst stage); and implanting the blastocyst into the uterus.

An embryo forms in the early development of a human (or other mammal). The use of embryos in IVF generally involves retrieval (harvesting) from the woman a number of oocytes resulting from a controlled ovarian stimulation cycle; fertilization (insemination of) one or more oocytes and culturing the fertilized egg (oocyte) for e.g. 3 days to form an embryo (i.e. allowing the fertilized oocyte to develop to the embryo stage); and implanting the embryo into the uterus.

Although the treatments described herein primarily are directed to IUI, in the case of over response (e.g., the production of a greater number of oocytes than desired for IUI), the oocytes may be harvested and used for IVF, including harvested and frozen for subsequent use in IVF (e.g., used in a “freeze-thaw” cycle” of an IVF protocol),

### Dosing Regimens

The serum concentration of anti-Müllerian hormone (AMH) is now established as a reliable marker of ovarian reserve. Decreasing levels of AMH are correlated with reduced ovarian response to gonadotrophins during COS. Further, high levels of AMH are a good predictor of excessive ovarian response, and an indicator of risk of OHSS. In the dosing regimens described herein, for the first (and, in some cases, subsequent) IUI treatment cycle, the individual daily dose is determined on the basis of the woman's serum AMH concentration (also referred to herein as “serum AMH level”) and, depending on her serum AMH concentration, her body weight. The dose is based on a recent determination of her serum AMH (i.e., determined within the last 24 months, including within the last 12 months), for example, measured by the ELECSYS® AMH Plus immunoassay (Roche), or similar assays such as ACCESS AMH Advanced from Beckman Coulter or UMIPULSE G AMH from Fujirebio. Additionally or alternatively, the serum concentration of anti-Müllerian hormone (AMH) in the patient may be determined using a Beckmann-Coulter Gen 2 assay as described in Arce et al., Fertility and Sterility 99:1644-53 (2013), or an equivalent method.

A subject may have a normal serum FSH level, e.g., a serum FSH level of 1 to 16 IU/L, for example 1 to 15 IU/L, for example 1 to 12 IU/L, for example 2 to 10 IU/L, all in the early follicular phase. Thus a composition or medicament or method as described herein may be for (use in) the treatment of infertility (and/or for controlled ovarian stimulation) in a subject having (or identified as having) a normal serum FSH level of 1 to 16 IU/L, for example 1 to 15 IU/L, for example 1 to 12 IU/L, for example 2 to 10 IU/L, all in the early follicular phase. Serum FSH may be measured by methods well known in the art, and optionally may be used to identify the patient for treatment.

A subject may have a BMI >15 and BMI <40 kg/m², for example a BMI >17.5 and BMI<38 kg/m², for example a BMI >18 and BMI <25 kg/m², for example a BMI >20 and BMI<25 kg/m². Thus a composition or medicament or method as described herein may be used for the treatment of infertility in a patient having BMI >1 and BMI <40 kg/m², for example a subject having BMI >17.5 and BMI <38 kg/m², for example a subject having BMI >18 and BMI <25 kg/m², for example a subject having BMI >20 and BMI <25 kg/m². Thus a composition or medicament or method as described herein may be used for the treatment of infertility in a patient having BMI >17.5 and BMI <32 kg/m². The patient (subject) may have a BMI of 30 kg/m² or over, for example a BMI of 30 to 40 kg/m². BMI may be measured by methods well known in the art, and optionally may be used to identify the patient for treatment.

In accordance with all aspects described herein, it is preferred that the treatment of infertility described herein, is or includes, a step of COS. In other words, the treatment of infertility is by controlled ovarian stimulation (as described herein) for intrauterine insemination. The cause of infertility could be the woman's partner suffering from male infertility, although it will be appreciated that according to the present invention it is the woman (female) who is treated by COS.

As used herein, “day one of treatment”, also referred to as “day one of stimulation”, refers to the first day that the dose of rFSH is administered to the patient. Day one of treatment (stimulation) may take place on day 1, 2 or 3, for example, on day 2 or day 3, of the patient's menstrual cycle. In other words, day one of treatment (stimulation) may be one, two or three days, for example, two or three days, after the patient commences menstrual bleeding, consistent with usage of this term in clinical practice with GnRH antagonist or GnRH agonist protocols. The term “during treatment” means on a day or on days that rFSH is being administered to the patient.

In the treatments, medicaments, methods and uses described herein, the administration of recombinant FSH (rFSH) starts on day one of treatment and may continue for two to twenty days, for example, may continue for 2, 3, 4, 5, 6, 7, 8, 9, 10, 11, 12, 13, 14, 15, 16, 17, 18, 19, or 20 days. The dose administered on day 1 is referred to herein as the “starting dose”. The administration of rFSH starts on day one of treatment and may continue for four to twenty days, for example, for seven to thirteen days, for example, for nine to thirteen days, for example, 10 to 13 days, for example, 10 to 11 days. The dose may be the same every day. However, variation of the dose depending on the patient's ovarian response (e.g., as measured by ultrasonography) is possible and, indeed, more likely.

Thus, a composition or medicament as described herein may be for, and a method or use as described herein may involve, administration at the defined dose of rFSH (e.g., the starting dose of rFSH) from day 1 to day 6 of treatment, that is, from day 1 to day 6 of stimulation. From day 7 of treatment (day 7 of stimulation) the dose of rFSH may be: increased (e.g. increased to a dose of up to 150% of the starting dose, for example increased to a dose of 120% to 150% of the starting dose); decreased (e.g. decreased to a dose of 50% of the starting dose, for example decreased to a dose of 50% to 80% of the starting dose); or maintained at the starting dose. The dose may be increased, decreased or maintained based on the patient's ovarian response (e.g., as measured by ultrasonography).

The doses (e.g., starting doses) disclosed herein may be the doses for a first cycle of IUI. For second and subsequent cycles of IUI, the “starting dose may be: increased (e.g. increased to a dose of up to 150% of the starting dose for the first cycle, for example increased to a dose of 120% to 150% of the starting dose for the first cycle); decreased (e.g. decreased to a dose of 50% of the starting dose for the first cycle, for example decreased to a dose of 50% to 80% of the starting dose for the first cycle); or maintained at the staring dose for the first cycle. The dose may be increased, decreased or maintained based on the patient's response to the first cycle (e.g. as measured by number of mature follicles ≥16 mm obtained in the first cycle on the day prior to or on the day of triggering, and/or, e.g., as detected in the last ultrasound scan before triggering).

As used herein “equivalent to” in relation to dosing means a dose that has the same pharmaceutical effect as the dose recited in the claim or herein. For example a dose of, or equivalent to, 0.05 μg per day may be the recited dose of 0.05 μg per day or may be a pharmaceutically equivalent dose such as a dose of 0.15 μg every 3 days. It will be appreciated this applies to any dose recited herein.

In accordance with all aspects described herein, the recombinant FSH may be human cell line-derived recombinant FSH as described in more detail below. In all aspects, the recombinant FSH may be that sold under the trademark REKOVELLE® (follitropin delta) (Ferring B. V.). In all aspects, the recombinant FSH may be administered by injection, e.g., subcutaneous injection.

Typically, in accordance with all aspects described herein, the recombinant FSH composition (e.g., pharmaceutical composition) or medicament is administered, or is for administration, prior to administration of a high (ovulatory) dose of human chorionic gonadotropin (hCG) (for example 4,000 to 11,000 IU hCG, e.g., 5,000 IU hCG, 10,000 IU hCG, etc.; or 150 to 500 μg recombinant hCG, for example 250 μg recombinant hCG); to induce final follicular maturation (e.g., to trigger ovulation). Thus, in some embodiments, the methods described herein further comprise administration of a high (ovulatory) dose of human chorionic gonadotropin (hCG).

The compositions, uses and methods described herein may be for, and may be effective for, intrauterine insemination in a patient treated for infertility.

The recombinant FSH doses listed herein may be for treatment of infertility in the patient's (subject's) first stimulation protocol (first stimulation “cycle”) by the methods and treatment protocols described herein. Thus, the composition(s) may be for use in the treatment of infertility in a patient (subject) who has not previously been treated for infertility by controlled ovarian stimulation; for use in the treatment of infertility in a patient (subject) who has not previously completed a treatment for infertility by controlled ovarian stimulation; or for use in the treatment of infertility in a patient (subject) who has not been treated for infertility by controlled ovarian stimulation in the previous six months, more preferably a patient (subject) who has not been treated for infertility by controlled ovarian stimulation in the previous twelve months. It will be appreciated that for further stimulation cycles (that is, treatments of infertility by controlled ovarian stimulation) by the methods and treatment protocols described herein, the doses may be adjusted according to actual ovarian response in the first cycle by the methods and treatment protocols described herein, such as outlined above.

### Treatments

In the proof-of-concept study described below, the applicants demonstrated the effectiveness of a personalised dose regimen for REKOVELLE® in IUI based on AMH and bodyweight. The percentage of patients treated who obtained 2 or 3 mature follicles (defined as a follicle ≥16 mm on the day prior to or on the day of triggering ovulation, and/or, e.g., as detected in the last ultrasound scan before triggering) in their first, second and third cycles was 34%, 37% and 47%, respectively. The percentage of patients who reached more than 3 follicles was 1.9%, 0% and 1.5%, respectively. Thus, the dosing regimen described herein achieves the desired goal. Only 6 multiple pregnancies out of 40 clinical pregnancies (15%) were observed, showing this is a safe approach. The clinical pregnancy rate per IUI cycle by intention to treat was 16%, 14.3% and 16.2% for the 3 IUI cycles, respectively. The cumulative clinical pregnancy rate over the 3 cycles among patients completing the study was 37.7%, indicative of usefulness for clinical practice.

In a first aspect, there is provided a composition (e.g., a pharmaceutical composition) comprising recombinant follicle stimulating hormone (rFSH) (optionally rFSH which includes α2,3- and α2,6-sialylation) for use in the treatment of infertility by intrauterine insemination (e.g., to increase pregnancy rate and/or live birth rate, e.g., to increase pregnancy rate and/or live birth rate compared to treatment with rFSH which includes only 2,3-sialylation, e.g., to increase pregnancy rate and/or live birth rate compared to fixed dose treatment with rFSH which includes only 2,3-sialylation) in a patient having serum AMH level of <15 pmol/L, wherein the composition is to be administered at a dose of, or equivalent to, 0.048 μg to 0.052 μg, for example 0.05 μg, rFSH per kg bodyweight of the patient per day, and corresponding methods of treatment. In an example, the composition is to be administered at a dose of 0.05 μg rFSH per kg bodyweight of the patient per day. The use or method may comprise a step of determining the serum AMH level of the patient (e.g., using the automated Elecsys® AMH assay from Roche Diagnostics or a similar method), and a step of administering the dose to a patient having serum AMH level of <15 pmol/L. The patient may be of age <35 years, age 35 to 40 years, or of age >40 years. The use or method may comprise a step of determining the age of the patient, and a step of administering the rFSH to a patient of age <35 years. The use may comprise a step of determining the age of the patient, and a step of administering the rFSH to a patient of age 35-40 years. The use may comprise a step of determining the age of the patient, and a step of administering the rFSH to a patient of age >40 years. The rFSH may be rFSH which includes α2,3- and α2,6-sialylation, for example recombinant FSH which includes α2,3- and α2,6-sialylation wherein 5% to 20% of the total sialylation is α2,6-sialylation and wherein 80% to 95% of the total sialylation is α2,3-sialylation. The rFSH may be rFSH which has been produced or expressed in a human cell line. The use or method may comprise a further step of triggering ovulation (e.g., by administering a dose of or 4,000 to 11,000 IU hCG). The use or method may comprise a further step of intrauterine insemination. The treatment of infertility by intrauterine insemination may be controlled ovarian stimulation for intrauterine insemination (e.g., controlled ovarian stimulation to prepare the patient for (a subsequent step of) intrauterine insemination). The treatment of infertility by intrauterine insemination may include controlled ovarian stimulation (e.g., using rFSH having α2,3- and α2,6-sialylation) with the aim of obtaining 2 or 3 mature follicles (defined as a follicle ≥16 mm on the day before or on the day of triggering ovulation, and/or, e.g., as detected in the last ultrasound scan before triggering) for a subsequent step of intrauterine insemination.

In a further aspect, there is provided a composition (e.g., a pharmaceutical composition) comprising recombinant follicle stimulating hormone (rFSH) (optionally rFSH which includes α2,3- and α2,6-sialylation) for use in the treatment of infertility by intrauterine insemination (e.g., to increase pregnancy rate and/or live birth rate, e.g., to increase pregnancy rate and/or live birth rate compared to treatment with rFSH which includes only 2,3-sialylation, e.g., to increase pregnancy rate and/or live birth rate compared to fixed dose treatment with rFSH which includes only 2,3-sialylation) in a patient having a serum AMH level of ≥15 pmol/L, wherein the composition is to be administered at a dose of, or equivalent to, 0.025 to 0.0475 μg rFSH per kg bodyweight of the patient per day, and corresponding methods of treatment. The patient may be a patient with polycystic ovary syndrome (PCOS). In another aspect, there is provided a composition (e.g., a pharmaceutical composition) comprising recombinant follicle stimulating hormone (rFSH) (optionally rFSH which includes α2,3- and α2,6-sialylation) for use in the treatment of infertility by intrauterine insemination (e.g., to increase pregnancy rate and/or live birth rate, e.g., to increase pregnancy rate and/or live birth rate compared to treatment with rFSH which includes only 2,3-sialylation, e.g., to increase pregnancy rate and/or live birth rate compared to fixed dose treatment with rFSH which includes only 2,3-sialylation) in a patient with PCOS having a serum AMH level of ≥15 pmol/L (preferably serum AMH of ≥23 pmol/L), wherein the composition is to be administered at a dose of, or equivalent to, 0.025 to 0.0475 μg rFSH per kg bodyweight of the patient per day, and corresponding methods of treatment. In another aspect, there is provided a composition (e.g., a pharmaceutical composition) comprising recombinant follicle stimulating hormone (rFSH) (optionally rFSH which includes α2,3- and α2,6-sialylation) for use in the treatment of infertility by intrauterine insemination (e.g., to increase pregnancy rate and/or live birth rate, e.g., to increase pregnancy rate and/or live birth rate compared to treatment with rFSH which includes only 2,3-sialylation, e.g., to increase pregnancy rate and/or live birth rate compared to fixed dose treatment with rFSH which includes only 2,3-sialylation) in a patient with PCOS [e.g. in a patient having a serum AMH level of ≥15 pmol/L (preferably serum AMH of ≥23 pmol/L)], wherein the composition is to be administered at a dose of, or equivalent to, 0.025 to 0.0475 μg rFSH per kg bodyweight of the patient per day, and corresponding methods of treatment. In an example, the composition is to be administered at a dose of 0.025 to 0.0475 μg rFSH per kg bodyweight of the patient per day.

The use or method may comprise a step of determining the serum AMH level of the patient (e.g., using the automated Elecsys® AMH assay from Roche Diagnostics or a similar method), and a step of administering the dose to a patient having serum AMH level of ≥15 pmol/L. The patient may be of age <35 years, age 35 to 40 years or of age >40 years. The use or method may comprise a step of determining the age of the patient, and a step of administering the recombinant FSH to a patient of age <35 years. The use or method may comprise a step of determining the age of the patient, and a step of administering the rFSH to a patient of age 35-40 years. The use or method may comprise a step of determining the age of the patient, and a step of administering the rFSH to a patient of age >40 years. The rFSH may be rFSH which includes α2,3- and α2,6-sialylation, for example rFSH which includes α2,3- and α2,6-sialylation wherein 5% to 20% of the total sialylation is α2,6-sialylation and wherein 80% to 95% of the total sialylation is α2,3-sialylation. The rFSH may be rFSH which has been produced or expressed in a human cell line. The use or method may comprise a further step of triggering ovulation (e.g., by administering a dose of 4,000 to 11,000 IU hCG). The use or method may comprise a further step of intrauterine insemination. The treatment of infertility by intrauterine insemination may be controlled ovarian stimulation for intrauterine insemination (e.g., controlled ovarian stimulation to prepare the patient for (a subsequent step of) intrauterine insemination). The treatment of infertility by intrauterine insemination may include controlled ovarian stimulation (e.g. using rFSH having α2,3- and α2,6-sialylation) with the aim of obtaining 2 or 3 mature follicles (defined as a follicle >16 mm on the day before or on the day of triggering ovulation, and/or, e.g., as detected in the last ultrasound scan before triggering) for a subsequent step of intrauterine insemination.

According to the present invention in another aspect there is provided a composition (e.g., a pharmaceutical composition) comprising follicle stimulating hormone (FSH) for use in the treatment of infertility by intrauterine insemination in a patient with PCOS, wherein the follicle stimulating hormone (FSH) includes alpha 2,3- and alpha 2,6 sialylation. Preferably, the FSH is a recombinant FSH which includes α2,3- and α2,6-sialylation wherein 5% to 20% of the total sialylation is α2,6-sialylation and wherein 80% to 95% of the total sialylation is α2,3-sialylation. The FSH may be a recombinant FSH which has been produced or expressed in a human cell line. The treatment of infertility by intrauterine insemination may be controlled ovarian stimulation for intrauterine insemination. The FSH may be for administration at a daily dose of 3 μg to 7 μg, preferably 3 μg to 5 μg. The patient may have a BMI of 30 40 kg/m² or over.

The patient with PCOS may have two or more of the following criteria (typically, other causes of menstrual disturbance and hyperandrogenism will have been excluded): Clinical and/or biochemical signs of hyperandrogenism; oligo-anovulation or anovulation; and polycystic ovaries, defined as the presence of 12 or more follicles (measuring 2-9 mm in diameter) in one or both ovaries and/or increased ovarian volume (more than 10 cm³). Alternatively or additionally, the patient with PCOS may have serum AMH of ≥23 pmol/L. The use or method may comprise a step of identifying (e.g. diagnosing) the patient as being a patient with PCOS. The use or method may comprise a step of identifying (e.g. diagnosing) the patient as being a patient with PCOS, and a step of administering the dose to a patient with PCOS. The patient may be identified as having PCOS (optionally by a medical practitioner) prior to treatment. The patient may be identified (optionally by a medical practitioner, e.g., prior to treatment) as having PCOS by assessing if they have two or more of the following criteria (and, typically, also ruling out other causes of menstrual disturbance and hyperandrogenism): Clinical and/or biochemical signs of hyperandrogenism; oligo-anovulation or anovulation; and polycystic ovaries, defined as the presence of 12 or more follicles (measuring 2-9 mm in diameter) in one or both ovaries and/or increased ovarian volume (more than 10 cm³). Alternatively or additionally, the patient may be identified as having PCOS by assessing whether they have serum AMH of ≥23 pmol/L. The serum AMH may be, for example, measured by the ELECSYS® AMH Plus immunoassay (Roche), or similar assays such as ACCESS AMH Advanced from Beckman Coulter or UMIPULSE G AMH from Fujirebio.

Additionally or alternatively, the serum concentration of anti-Müllerian hormone (AMH) in the patient may be determined using a Beckmann-Coulter Gen 2 assay as described in Arce et al., Fertility and Sterility 99:1644-53 (2013), or an equivalent method. The AMH should be determined within the 24 months, including within the 12 months, prior to treatment. A serum AMH of ≥23 pmol/L is indicative of PCOS.

The composition may be for administration, and the method may comprise administration, at the defined dose of rFSH (e.g., the starting dose of rFSH) from day 1 to day 6 of treatment, that is from day 1 to day 6 of stimulation. From day 7 of treatment (day 7 of stimulation) the dose of rFSH may be: decreased (e.g. decreased to a dose of 50% of the starting dose, for example decreased to a dose of 50% to 80% of the starting dose); or maintained at the staring dose. In other embodiments, from day 7 of treatment (day 7 of stimulation), the dose of FSH may be increased (e.g. increased to a dose of up to 150% of the starting dose, for example increased to a dose of 120% to 150% of the starting dose); The dose may be increased, decreased or maintained based on the patient's ovarian response (e.g., as measured by ultrasonography).

The doses (e.g., starting doses) disclosed herein may be the doses for a first cycle of IUI. For second and subsequent cycles of IUI, the “starting dose may be: increased (e.g., increased to a dose of up to 150% of the starting dose for the first cycle, for example increased to a dose of 120% to 150% of the starting dose for the first cycle, for example increased to a dose of 125% or 150% of the dose for the starting cycle); decreased (e.g. decreased to a dose of 50% of the starting dose for the first cycle, for example decreased to a dose of 50% to 80% (e.g. 67%) of the starting dose for the first cycle); or maintained at the staring dose for the first cycle. The dose may be increased, decreased or maintained based on the patient's response to the first cycle (e.g. as measured by number of mature follicles ≥ 16 mm obtained in the first cycle, e.g. on the day prior to or on the day of triggering, and/or e.g. as detected in the last ultrasound scan before triggering).

In some examples, for third and subsequent cycles of IUI, the “starting dose” may be: increased (e.g., increased to a dose of up to 150% of the starting dose for the second cycle, for example, increased to a dose of 120% to 150% of the starting dose for the second cycle, for example, increased to a dose of 125% or 150% of the dose for the second cycle); decreased (e.g., decreased to a dose of 50% of the starting dose for the second cycle, for example, decreased to a dose of 50% to 80% (e.g. 67%, 80%) of the starting dose for the second cycle); or maintained at the staring dose for the second cycle. The dose may be increased, decreased or maintained based on the patient's ovarian response to the second cycle (e.g., as measured by number of mature follicles ≥16 mm obtained in the second cycle, e.g., on the day prior to or on the day of triggering, and/or e.g. as detected in the last ultrasound scan before triggering).

As noted above, typically, the recombinant FSH composition (e.g., pharmaceutical composition) or medicament is administered, or is for administration, prior to administration of a high (ovulatory) dose of human chorionic gonadotropin (hCG) (for example, 4,000 to 11,000 IU hCG, e.g., 5,000 IU hCG, 10,000 IU hCG, etc.; or 150 to 500 μg recombinant hCG, for example 250 μg recombinant hCG); to induce final follicular maturation. In some embodiments, therefore, the methods described herein further comprise administration of a high (ovulatory) dose of human chorionic gonadotropin (hCG).

Additionally, or alternatively, the treatment may further comprise monitoring and/or control of over-response to treatment (e.g., OHSS).

Recombinant FSH and rFSH Compositions

As noted above, the methods and compositions described herein use recombinant FSH (rFSH). FSH comprises a 92 amino acid alpha sub-unit, also common to the other glycoprotein hormones LH and chorionic gonadotropin (CG), and a 111 amino acid beta sub-unit unique to FSH that confers the biological specificity of the hormone (Pierce and Parsons, 1981). Each sub-unit is post translationally modified by the addition of complex carbohydrate residues. Both subunits carry 2 sites for N-linked glycan attachment, the alpha sub-unit at amino acids 52 and 78 and the beta sub-unit at amino acid residues 7 and 24. FSH is thus glycosylated to about 30% by mass.

The glycosylation of rFSH products reflects the range of glycosyl-transferases present in the host cell line. Commercially available rFSH products derived from engineered Chinese Hamster Ovary (CHO) cells have a more limited range of glycan modifications than those found on the natural products. Examples of the reduced glycan heterogeneity found in CHO cell derived rFSH include a lack of bisecting glucosamine and a reduced content of core fucosylation and acetyl lactosamine extensions. In addition, CHO cells are only able to add sialic acid using the α2,3 linkage; CHO cell-derived rFSH only includes α2,3-linked sialic acid and does not include α2,6-linked sialic acid. Thus, CHO cell-derived rFSH is different from naturally produced FSH (e.g., human pituitary/serum/urinary FSH) which contains glycans with a mixture of α2,3 and α2,6-linked sialic acid, with a predominance of the former.

As noted above, the present applicants have developed a human cell line-derived rFSH which is the subject of International Patent Application No. PCT/GB2009/000978, published as WO2009/127826A, and also approved by the EC as REVOKELLE® (follitropin delta, also known as FE 999049). Recombinant FSH with a mixture of both α2,3 and α2,6-linked sialic acid was made by engineering a human cell line to express both rFSH and α2,3 sialyltransferase. The amino acid sequence of the human cell line-derived recombinant FSH which is the subject of International Patent Application No. PCT/GB2009/000978, published as WO2009/127826A (e.g., FE 999049), is the native human FSH sequence, but the product has a different glycosylation pattern. The expressed product is highly acidic and carries a mix of both α2,3- and α2,6-linked sialic acids; the latter provided by the endogenous sialyltransferase activity. It was found that the type of sialic acid linkage, α2,3- or α2,6-, can have a dramatic influence on biological clearance of FSH. Thus, REVOKELLE® (e.g., FE 999049) may be more biologically appropriate compared to CHO cell-derived recombinant products that have only α2,3 linked sialic acid and have decreased sialic acid content.

Thus, the recombinant FSH used in accordance with the methods and compositions described herein may be produced or expressed in a human cell line, such as a PER.C6® cell line. The recombinant FSH may be produced or expressed in a PER.C6® cell line (deposited under ECACC deposit No. 96022940), a PER.C6® derived cell line or a modified PER.C6® cell line. Recombinant FSH which is produced or expressed in a PER.C6® cell line will include some α2,6-linked sialic acids (α2,6 sialylation) provided by endogenous sialyltransferase activity (of the cell line) and will include some α2,3-linked sialic acids (α2,3 sialylation) provided by endogenous sialyl transferase activity. The cell line may be modified using α2,3-sialyltransferase. The cell line may be modified using α2,6-sialyltransferase. Alternatively or additionally, the recombinant FSH may include α2,6-linked sialic acids (α2,6 sialylation) provided by endogenous sialyl transferase activity (of the cell line).

The recombinant FSH used in the methods and compositions described herein may include α2,3- and α2,6-sialylation. The recombinant FSH for use according to the invention may have 1% to 99% of the total sialylation being α2,3-sialylation. The recombinant FSH for use according to the invention may have 1% to 99% of the total sialylation being α2,6-sialylation. The recombinant FSH may have 1% to 50% of the total sialylation as α2,6-sialyation, and 50% to 99% of the total sialylation as α2,3-sialyation. For example, 80% to 95%, for example 80% to 90%, for example 82% to 89%, for example 85% to 89% of the total sialylation may be α2,3-sialylation. For example, 5% to 20%, for example 10% to 20%, for example 11% to 18%, for example 11% to 15%, of the total sialylation may be 2,6-sialylation. In an example, the recombinant FSH has 5% to 20% of the total sialylation as α2,6-sialyation, and 80% to 95% of the total sialylation as α2,3-sialyation. In another example, the recombinant FSH has 50% to 80% of the total sialylation as α2,6-sialyation, and 20% to 50% of the total sialylation as a 2,3-sialyation.

Herein, by “sialylation”, it is meant the amount of sialic residues present on the recombinant FSH carbohydrate structures. Consistent with usage in the art, α2,3-sialylation means sialylation at the 2,3 position and α2,6 sialylation means sialylation at the 2,6 position. Thus “% of the total sialylation may be α2,3 sialylation” refers to the % of the total number of sialic acid residues present in the FSH (or hCG) which are sialylated in the 2,3 position. The term “% of the total sialylation being 2,6-sialylation” refers to the % of the total number of sialic acid residues present in the FSH (or hCG) which are sialylated in the 2,6 position.

In all aspects, the rFSH may be present as a single isoform or as a mixture of isoforms.

The composition may be a pharmaceutical composition. The pharmaceutical composition is for the treatment of infertility. The treatment of infertility may comprise COS prior to IUI. The pharmaceutical composition may be used, for example, in medical indications where known FSH preparations are used in IUI, in accordance with the methods and treatment protocols disclosed herein

The recombinant FSH, composition, or pharmaceutical composition can be formulated into well-known compositions for subcutaneous administration (e.g., for subcutaneous injection). A typical composition comprises a pharmaceutically acceptable carrier, such as aqueous solution, nontoxic excipients, including salts and preservatives, buffers and the like, as described in Remington's Pharmaceutical Sciences fifteenth edition (Matt Publishing Company, 1975), at pages 1405 to 1412 and 1461 to 1487, and the national formulary XIV fourteenth edition (American Pharmaceutical Association, 1975), among others. For example, the recombinant FSH, composition or pharmaceutical composition can be formulated for injection, such as for subcutaneous injection.

Examples of suitable aqueous and non-aqueous pharmaceutical carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate.

The compositions of the present invention may also comprise additives such as but not limited to preservatives, wetting agents, emulsifying agents, surfactants and dispersing agents. Antibacterial and antifungal agents can be included to prevent growth of microbes and includes, for example, m-cresol, benzyl alcohol, paraben, chlorobutanol, phenol, sorbic acid, and the like. If a preservative is included, benzyl alcohol, phenol and/or m-cresol are preferred; however, the preservative is by no means limited to these examples. Furthermore, it may be desirable to include isotonic agents such as sugars, sodium chloride, amino acids and the like.

For example, the composition or medicament may comprise recombinant FSH and one or more of polysorbate 20, L-methionine, phenol, and arginine hydrochloride. Such a composition may be formulated for injection, such as for subcutaneous injection. For example, the composition or medicament may be the REKOVELLE® formulation (rFSH with excipients phenol, polysorbate 20, L-methionine, sodium sulphate decahydrate, disodium phosphate dodecahydrate, phosphoric acid [concentrated, for pH-adjustment], sodium hydroxide [for pH-adjustment], and water for injection).

Injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use. Injectable formulations can be supplied in any suitable container, e.g., vial, pre-filled syringe, injection cartridges, and the like.

The recombinant FSH, composition, or medicament may be formulated for single use or for multiple use (multiple dose). If the recombinant FSH, composition, or medicament is formulated for multiple use, typically one or more preservatives is included. If a preservative is included, benzyl alcohol, phenol or m-cresol, are preferred; however, the preservative is by no means limited to these examples. The single use or multiple use formulated composition or medicament may further comprise an amino acid or combination of amino acids. Typically, the amino acid is arginine, for example added as arginine or more typically arginine hydrochloride.

The recombinant FSH, composition, or medicament may be included in a container such as a vial, prefilled cartridge (e.g., for single administration or multiple use) or an injection device such as a “pen” for e.g., administration of multiple doses.

The recombinant FSH, composition or pharmaceutical composition may be a formulation (e.g., injectable formulation) including rFSH.

The recombinant FSH, composition or medicament can be supplied in any appropriate package. For example, a composition or medicament can include a number of containers (e.g., pre-filled syringes or vials) containing FSH. The syringes or vials may be packaged in a blister package or other means to maintain sterility. Any composition or medicament can optionally include instructions for using the FSH formulation.

The pH and exact concentration of the various components of the pharmaceutical composition may be adjusted in accordance with routine practice in this field. See GOODMAN and GILMAN's THE PHARMACOLOGICAL BASIS FOR THERAPEUTICES, 7^{th} ed. In a typical embodiment, the recombinant FSH, composition or medicament are supplied as compositions for parenteral administration. General methods for the preparation of the parenteral formulations are known in the art and are described in REMINGTON; THE SCIENCE AND PRACTICE OF PHARMACY, supra, at pages 780-820. The parenteral compositions can be supplied in a liquid formulation or as a solid which will be mixed with a sterile injectable medium just prior to administration. The parenteral compositions may be supplied in dosage unit form for ease of administration and uniformity of dosage.

In a further aspect there is provided the use of recombinant follicle stimulating hormone (FSH) in the manufacture of a medicament for the uses as described and claimed herein.

Further aspects are illustrated in the following examples, which are not limiting in any respect.

### EXAMPLES

The following examples use REKOVELLE®, follitropin delta, which is a recombinant FSH expressed in a PER.C6® cell line engineered by the methods disclosed in WO2013/020996 and WO2009/127826A.

The Marketing Authorisation holder for REKOVELLE® is Ferring Pharmaceuticals A/S of Kay Fiskers Plads 11, 2300 Copenhagen S, Denmark, and REKOVELLE® is available in the UK from Ferring Pharmaceuticals of Drayton Hall, Church Road, West Drayton, UB7 7PS, UK.

The active substance in REKOVELLE® is follitropin delta (FE999049). REKOVELLE® is highly sialylated and includes α2,3- and α2,6-sialylation, with about 85% to 90% of the total sialylation being α2,3-sialylation and about 10% to 15% of the total sialylation being α2,6-sialylation.

REKOVELLE® is a clear and colourless solution for injection (injection). One mL of solution contains 33.3 micrograms of follitropin delta in each mL of solution. The other ingredients are phenol, polysorbate 20, L-methionine, sodium sulphate decahydrate, disodium phosphate dodecahydrate, concentrated phosphoric acid, sodium hydroxide and water for injections.

### Example 1: Personalized IUI Treatment Study (PITS): a Proof of Concept Study Evaluating the Effect of Personalized Dosages of Follitropin Delta in Intrauterine Insemination

### Clinical Trial Registration Number: NCT03830723

### Introduction

The PITS study aimed to evaluate the effect of follitropin delta on the ovarian response using an individualized dosing regimen in controlled ovarian stimulation for IUI. The secondary objectives were the incidence of cancelled cycles, multiple pregnancies, and the cumulative clinical pregnancy rate over 3 consecutive cycles.

### Methods

### Design

This was a single center, open-label, single-cohort prospective study assessing the efficacy and safety of a personalized dosage of follitropin delta for intrauterine insemination. Patients were enrolled at one center in Canada to receive up to 3 cycles of IUI. The study protocol was approved by local regulatory authorities, as well as independent ethics committee. The study was performed in accordance with the principals of the Declaration of Helsinki, the International Conference on Harmonization Guidelines for Good Clinical Practice, and local regulatory requirements. All participants provided written and informed consent before undergoing any study-related procedures.

### Participants

Women enrolled in the study were between 18 and 42 years of age, had at least one permeable Fallopian tube confirmed by laparoscopy, hysterosalpingography (HSG), or hysterosalpingosonography (HSSG), or one pregnancy in the last 3 years, and were followed at the fertility center. Additional inclusion criteria were regular menstrual cycles of from 26 to 39 days, presence of both ovaries, use of ejaculated sperm (fresh or frozen) from partner or donor, and semen analysis considered adequate for IUI in accordance with the center's standard practice. The main exclusion criteria were high risk for OHSS (AMH ≥35 pmol/L (4.9 ng/ml)), body weight >100 Kg, history of severe malformation (unicornuate or bicornuate uterus) or uterine anomaly including fibroids ≥5 cm, uncontrolled thyroid or adrenal dysfunction, pituitary tumour or persistent ovarian cysts or enlargement not due to PCOS >3 cm. Other exclusion criteria were the use of hormone treatment in the last 3 months prior to start of stimulation, the diagnosis of hydrosalpinx, malignancies, breast pathology incompatible with gonadotropin stimulation, hypersensitivity to follitropin delta or to any ingredient in the formulation, and the addition of other infertility medication that can influence follicle stimulation and maturation such as growth hormone (GH).

### Procedures

All subjects participating in the study were treated with a personalized daily subcutaneous dose of follitropin delta (Rekovelle®; Ferring Pharmaceuticals) for the first IUI cycle, determined based on body weight and serum AMH level measured within the last 24 months before the start of ovarian stimulation (Table 1).

TABLE 1
Follitorpin delta Dosing for IUI

| Measured AMH | Rounded AMH | AMH | | |
|---|---|---|---|---|
| pmol/L | pmol/L | ng/mL | Dose for IUI | |
| <14.5 | <15 | <2.1 | 0.05 | μg/kg |
| 14.5-16.4 | 15-16 | 2.1-2.24 | 0.0475 | μg/kg |
| 16.5-17.4 | 17 | 2.38 | 0.045 | μg/kg |
| 17.5-18.4 | 18 | 2.52 | 0.0425 | μg/kg |
| 18.5-20.4 | 19-20 | 2.66-2.8 | 0.04 | μg/kg |
| 20.5-22.4 | 21-22 | 2.94-3.08 | 0.0375 | μg/kg |
| 22.5-24.4 | 23-24 | 3.22-3.36 | 0.035 | μg/kg |
| 24.5-27.4 | 25-27 | 3.5-3.78 | 0.0325 | μg/kg |
| 27.5-32.4 | 28-32 | 3.92-4.48 | 0.03 | μg/kg |
| 32.5-39.4 | 33-39 | 4.62-5.46 | 0.0275 | μg/kg |
| >39.5 | >40 | >5.6 | 0.025 | μg/kg |

These doses were designed to achieve 2-3 mature follicles for IUI.

For the 2^{nd} and 3^{rd} cycles, the dose of follitropin delta was adjusted by taking into consideration the number of mature follicles visualized at the last ultrasound (by transvaginal ultrasound (TVU)) before the insemination and the participant's age (Table 2 and Table 3), even if adjustments were done during the first cycle (see Table 5). Additionally, if there were more than 4 follicles having a diameter of 13.0-15.9 mm, a reduction of follitropin delta dose by 33% from the prescribed initial dose was done to decrease the risk of hyper response in the next cycle.

TABLE 2
rFSH Dose Customisation for IUI (2^{nd} cycle)

| | | <35 | 35-40 | >40 |
|---|---|---|---|---|
| | | years old | years old | years old |
| Num. | 1 follicle | Initial dose | ID + 25% | ID + 50% |
| follicles ≥16 mm | | (ID) + 25% | | |
| at last TVU | 2 follicles | ID | ID | ID |
| prior to IUI | 3 follicles | ID − 20% | ID | ID |
| | ≥4 follicles* | ID − 33% | ID − 33% | ID − 33% |

*Previous cycle would have been cancelled as per cancelation criteria

TABLE 3
rFSH Dose Customisation for IUI (3^{rd} cycle)

| | | <35 | 35-40 | >40 |
|---|---|---|---|---|
| | | years old | years old | years old |
| Num. | 1 follicle | 2nd dose + | 2nd dose + | 2nd dose + |
| follicles ≥16 | | 25% | 50% | 50% |
| mm at | 2 follicles | 2nd dose | 2nd dose | 2nd dose |
| last TVU | 3 follicles | 2nd dose | 2nd dose | 2nd dose |
| prior to IUI | ≥4 follicles* | 2nd dose − | 2nd dose − | 2nd dose − |
| | | 33% | 33% | 20% |

*Previous cycle would have been cancelled as per cancelation criteria

Follitropin delta therapy was initiated on day 3 of the menstrual cycle and the dose was kept the same for the first 5 days of stimulation. The response to stimulation was monitored via vaginal sonography (TVU), starting at day 8 (day 6 of stimulation). After day 6 of stimulation, participants underwent transvaginal ultrasound examinations every 2-3 days (according to an expected growth of 2 mm/day of follicles ≥12 mm). The follitropin delta dose adjustments during the cycle were done according to predefined criteria described in Table 4. The size of the follicles was calculated from the measurements of the two maximum diameters of the follicle (two-dimensional size).

TABLE 4
In-Cycle IUI rFSH Dose Adjustments

| | |
|---|---|
| If more than 3 follicles ≥11 mm | Decrease rFSH dose by 20% |
| If at Day 15 of stimulation | Consider cycle cancelation |
| there are no follicles >11 mm | |
| In all other cases | Continue same dose of rFSH |

Targeted ovarian response was defined as 2 to 3 follicles ≥16 mm on the day of the trigger or one day before and/or, e.g., as detected in the last ultrasound scan before triggering. If only 1 mature follicle had developed, the physicians proceeded with the IUI as per the clinic's standard of care. If more than 3 mature follicles have developed, a cancelation or a conversion to IVF were considered.

The triggering medication was choriogonadotropin alfa (Ovidrel®; EMD Serono) and was administered when clinically indicated. Luteal phase support with 100 mg progesterone vaginal tablets (Endometrin®; Ferring Pharmaceuticals) twice daily was started on the day of the IUI for all women participating in the study until day 14 when the urine pregnancy test was performed.

### Definitions

Positive pregnancy test was defined as a positive urine pregnancy test. Clinical pregnancy was defined as a viable intrauterine pregnancy at 6-8 weeks confirmed by an ultrasound scan. Ongoing pregnancy was defined as a viable intrauterine pregnancy of at least 12 weeks duration confirmed on an ultrasound scan. Pregnancy loss was defined as the spontaneous demise of a pregnancy before 24 weeks of gestational age. Multiple pregnancy rate was defined as visualisation of two or more embryos with a fetal heartbeat at the viability ultrasound between 6 and 8 weeks of pregnancy per total number of clinical pregnancies.

### Outcomes

The co-primary endpoints of this study included the ovarian response (specifically the proportion of cycles in which 2 or 3 mature follicles were reached), and the clinical pregnancy rate. The cumulative clinical pregnancy rate in up to three IUI cycles, as well as the proportion of subjects with multiple pregnancies, were also studied as secondary endpoints.

Other secondary endpoints included duration of stimulation, total medication dose, medication dose adjustment and endometrial thickness.

Safety endpoints included the proportion of patients with cancelled cycles and the reasons for cycle cancellation, the incidence of early OHSS (including OHSS of moderate/severe grade), and the rate of conversion to IVF.

### Statistical Methods

This study employed a single-cohort, prospective, open-label design. Continuous variables were assessed for normality of distribution and reported as means and standard deviations. Differences between continuous variables were assessed by T-test or ANOVA and multivariate regression analysis for normally distributed variables or by Wilcoxon signed-rank test for non-normally distributed variables. Correlation between continuous variables were assessed by Pearson's correlation test. Effect size was assessed by Cohen's d test. Categorical variables are expressed as a percentage and were compared by Chi-square test or multivariate regression analysis. Results for pregnancy outcome were assessed by intention to treat.

The effect of age on treatment efficacy was assessed by the differences in the pregnancy outcomes between patients classified in the following age groups: <35 years, 35-40 years and >40 years. ANOVA was used to assess the differences between the different age cohorts for continuous variables and multivariate logistic regression for categorical variables. Statistical tests were performed using SPSS version 26 and R for Cohen's d test.

### Results

The mean age of all subjects was 34.46 years (±4.5), stratified as following: <35 years (47.7%), 35-40 years (38.5%) and >40 years (13.8%). The mean weight was 69.2 kg (±11.24), mean serum AMH level was 3.84 ng/ml (±13), mean serum FSH level was 6.3 IU (±2.61), and mean antral follicular count was 15 (±8.6). Primary infertility was present in 54.5% of the participants and the mean of years of infertility was 2.48 (±3.57). The causes of infertility were unexplained in 41.8%, female factor in 19.1%, male factor in 26.3% and other in 12.8% of the enrolled participants.

The clinical pregnancy rate per IUI cycle by intention to treat (ITT) was 16%, 14.3% and 16.2%, respectively (Table 5). The cumulative clinical pregnancy rate for patients who received up to 3 IUI cycles was 37.7% (Table 6). The cumulative live birth rate by ITT was 33% (Table 7) (calculated ad hoc, as it was not part of the study protocol). Only 6 multiple pregnancies over 40 pregnancies (15%) were observed. These were all twin gestations. Multiple pregnancy rate per IUI cycle was 12.5%, 8.3% and 27.3%, respectively (Table 7).

TABLE 5
Pregnancy outcomes

| | | | | | |
|---|---|---|---|---|---|
| | 1^{st} cycle | 2^{nd} cycle | 3^{rd} cycle | | P |
| Characteristic | (n = 106) | (n = 84) | (n = 68) | OVERALL | value |

PREGNANCY OUTCOMES

| | | | | | |
|---|---|---|---|---|---|
| Positive | 22 | 12 | 13 | 18.2% | 0.11 |
| pregnancy test | (20.2%) | (14.3%) | (19.1%) | (47/258) | |
| Biochemical | 5 | 0 | 2 | 2.7% | 0.21 |
| pregnancy | (4.7%) | (0%) | (2.9%) | (7/258) | |
| Clinical | 16 | 12 | 11 | 15.1% | 0.36 |
| pregnancy | (16.0%) | (14.3%) | (16.2%) | (39/258) | |
| Ongoing | 15 | 12 | 9 | 13.95% | 0.08 |
| pregnancy | (14.15%) | (14.28%) | (13.23%) | (36/258) | |
| Live birth | 15 | 11 | 9 | 13.56% | 0.29 |
| | (14.15%) | (13.09%) | (13.23%) | (35/258) | |
| Early | 2 | 0 | 2 | 1.6% | 0.40 |
| miscarriage | (1.8%) | (0%) | (2.9%) | (4/258) | |
| rate (Before | | | | | |
| 12 weeks) | | | | | |

Results per intention to treat.
Note:
Values are mean +/− standard deviation or numbers and percentages unless otherwise indicated.
NA = not available NS = not statistically significant.

TABLE 6
Cumulative pregnancy outcomes up to 3 cycles.

| | Cumulative rate up to 3 cycles |
|---|---|
| Positive pregnancy test | 44.34% |
| Clinical pregnancy | 37.73% |
| Ongoing pregnancy | 33.96% |
| Live birth | 33.02% |

TABLE 7
Safety endpoints

| | | | | | |
|---|---|---|---|---|---|
| | 1^{st} cycle | 2^{nd} cycle | 3^{rd} cycle | | P |
| Characteristic | (n = 106) | (n = 84) | (n = 68) | OVERALL | value |

SAFETY MEASURES

| | | | | | |
|---|---|---|---|---|---|
| Proportion of | 9 | 2 | 6 | 6.6% | 0.29 |
| patients with | (8.5%) | (2.4%) | (8.9%) | (17/258) | |
| cancelled | | | | | |
| cycles | | | | | |
| Multiple | 2 | 1 | 3 | 2.3% | 0.21 |
| pregnancy rate | (1.9%) | (1.2%) | (4.4%) | (6/258) | |
| per cycle | | | | | |
| Multiple | 2 | 1 | 3 | 15% | 0.12 |
| pregnancy rate | (12.5%) | (8.3%) | (27.3%) | (6/40) | |
| per clinical | | | | | |
| pregnancy | | | | | |

The percentage of patients who obtained 2 or 3 follicles in the first, second and third cycles, was 34%, 37% and 47%, respectively, and the mean number of mature follicles was 1.4, 1.4 and 1.6, respectively. The percentage of patients who reached more than 3 follicles in the first, second and third cycles was 1.9%, 0% and 1.5%, respectively, demonstrating the safety of the follitropin delta dosing regimen. The proportion of patients with cycle cancelation in the first, second and third cycles was 8.5%, 2.4% and 8.8%, respectively (Table 7).

The reasons for the 17 cancellations during the 3 IUI cycles were: 7-no response, 4-excessive response, 2-premature ovulation, 2-ovarian cyst and 2-patient withdrawal from the study. There was no OHSS reported and no conversion to IVF throughout the 3 IUI cycles.

Statistically significant differences were observed in the initial dose of follitropin delta between the first (3.25 μg±0.74) and the third (5.20 μg±1.89) IUI cycle. The mean total amount of follitropin delta used for each cycle was 27.7 μg (±12.7), 31.4 μg (±12.7) and 40.1 μg (±19.1), respectively. This was significantly different from the first to the second and from the second to the third IUI cycle (P<0.05). Between cycles, follitropin delta dose adjustment was on average the following: From the first to the second IUI cycle: +25% of the initial dose (44%) and +50% of the initial dose (6%), and from the second to the third IUI cycle: +25% (20%) and +50% (24%) (Table 8). There was no significant difference in the mean number of days of follitropin delta stimulation for each IUI cycle: 8.5 (+3.0), 7.9 (+2.6) and 7.8 (+2.5), respectively.

TABLE 8
Ovarian response outcomes

| | | | | |
|---|---|---|---|---|
| | 1^{st} cycle | 2^{nd} cycle | 3^{rd} cycle | |
| Characteristic | (n = 106) | (n = 84) | (n = 68) | P value |

OVARIAN REPONSE

| Number of cycles with 2-3 | 36 | (34%) | 31 | (37%) | 32 | (47%) | 0.044 |
|---|---|---|---|---|---|---|---|
| follicles | | | | | | | |
| Number of mature follicles | | | | | | | |
| obtained per cycle | | | | | | | |
| 0 | 4 | (3.8%) | 2 | (2.4%) | 3 | (4.4%) | — |
| 1 | 64 | (60.4%) | 51 | (60.7%) | 32 | (47.1%) | |
| 2 | 30 | (28.3%) | 26 | (31%) | 20 | (29.4%) | |
| 3 | 6 | (5.7%) | 5 | (6%) | 12 | (17.6%) | |
| 4 | 2 | (1.9%) | 0 | (0%) | 1 | (1.5%) | |
| >4 | 0 | (0%) | 0 | (0%) | 0 | (0%) | |
| Duration of stimulation | 8.59 ± 3.06 | | 7.98 ± 2.64 | | 7.82 ± 2.5 | | 0.06 |
| (days) | | | | | | | |
| Endometrial thickness (mm) | 9.26 ± 2.29 | | 9.51 ± 2.05 | | 10.1 ± 2.7 | | 0.036 |
| Initial dose of follitropin | 3.25 ± 0.74 | | 3.99 ± 1.06 | | 5.20 ± 1.89 | | <0.0001 |
| delta | | | | | | | |
| Dose adjustment during | 6 | (5.6%) | 12 | (14.3%) | 12 | (17.6%) | |

| | | | | |
|---|---|---|---|---|
| cycle (decrease by 20%) | | | | |
| Total dose per cycle | 27.75 ± 12.7 | 31.37 ± 12.57 | 40.06 ± 19.10 | <0.0001 |

Note:
Values are mean +/− standard deviation or numbers and percentages unless otherwise indicated.
NA = not available NS = not statistically significant.

Endometrial thickness increased from the first to the second and third IUI cycle with means of 9.3 mm (±2.1), 9.5 mm (±2.0) and 10.1 mm (±2.7) per cycle, respectively, with a significant difference (P=0.04) between the first and the third IUI cycle. (Table 8).

There was no statistically significant correlation between the total amount of follitropin delta administered per cycle, the number of mature follicles, and the clinical pregnancy rate. There was, however, a trend towards a higher number of mature follicles, and higher clinical pregnancy rate and multiple pregnancy rate with the higher follitropin delta amount used in the third IUI cycle.

### Discussion

This is the first study to evaluate the use of follitropin delta for ovarian stimulation prior to intrauterine insemination. The initial dose of follitropin delta for IUI was determined as shown in Table 1.

Gonadotropin doses (e.g., FSH doses) for IUI and IVF are not comparable, at least because the number of mature follicles expected (desired) is not the same, because of the risk of multiple pregnancies in IUI. To achieve 2-3 mature follicles in this trial, the doses used were about one fourth of those used for IVF. This is a relatively conservative (low) dose for a first cycle designed to be clinically effective (e.g., to provide the desired amount of 2-3 mature follicles) for all patients, but was considered to be unlikely to result in multiple pregnancy in the first cycle. This conservative and safer dosage for the first insemination allows better personalization of the doses for the second and third IUI cycles.

For the second and third cycles, the follitropin delta dose was adjusted taking into consideration the number of mature follicles reached during the previous insemination cycle and the age of the participant (see Tables above). This strengthened the individualized dosing approach. For example, oocyte quality generally decreases with age, and the dosing regimen described herein may compensate quality with quantity.

The treatments described herein compare favorable with previously reported studies using FSH in IUI.

Gregoriou O, et al., Fertil. Steril. 2008; 90:678-683, randomized 50 couples (mean age 32 years) who had previously failed three cycles of clomiphene citrate/IUI to receive either FSH/IUI (Gonal F or Puregon, CHO-cell line derived recombinant FSH at a dose of 150 IU every two days) or letrozole/IUI for a maximum of three cycles. The cumulative live birth rate was 28% for the FSH/IUI group and 20% for the letrozole/IUI group. In contrast, in the PITS study reported herein, the live birth rate observed was higher (33% vs 28%).

Three other randomized clinical trials have compared clomiphene citrate (CC)/IUI with gonadotropin (CHO cell line derived recombinant FSH)/IUI. Berker B, et al., Arch. Gynecol. Obstet. 2011; 284:1561-1566, randomized 93 couples. Ongoing pregnancy rate per cycle was 9.6% with CC/IUI and 15.6% with gonadotropin/IUI, with a multiple pregnancy rate of 13.3% in the gonadotropin/IUI group. Dankert T, et al., Hum. Reprod. 2007; 22:792-797, randomized 68 couples, and after four cycles of IUI the cumulative live birth rate was 28.2% in CC/IUI group and 26.9% in the gonadotropin (Gonal F)/IUI group, although there was a higher cancellation rate (primarily for over-response) in the gonadotropin/IUI group (8.7%). The AMIGOS trial reported in Diamond MP, et al., Fertil. Steril. 2015; 103(4): 962-973, compared gonadotropin (follitropin alfa), estrogen antagonist, and aromatase inhibitor treatment in up to 4 IUI cycles. The cumulative clinical pregnancy rate was 35.5%, 28.3% and 22.4%, and the cumulative live birth rate was 32.2%, 23.3% and 18.7%, respectively, for these 3 categories of agents. The multiple pregnancy rate in the AMIGOS trial was 31.8%, 9.4% and 13.4%, respectively, including 24 twins and 10 triplets in the gonadotropin/IUI group.

In the PITS study reported herein, a higher cumulative clinical pregnancy rate (37.7%) and cumulative live birth rate (33%) were obtained compared to these three randomized clinical trials, and that improvement was achieved with only three cycles of IUI instead of four. Additionally, the multiple pregnancy rate in the PITS study (15% with no triplets) was half that observed in the AMIGOS trial (31.8%) with a lower cancelation rate than that observed in the Dankert's trial. These results support the safety of the dosing regimen disclosed herein. While not wanting to be bound by theory, the use of a novel, human-derived recombinant FSH (follitropin delta), which has been shown to produce a milder ovarian stimulation in polycystic ovaries (PCO) patients, may be partly responsible for the reduced multiple pregnancy rate.

The PITS study showed that a personalised approach using individualized dosing of follitropin delta results in a higher clinical pregnancy rate (than, e.g., with use of, e.g., a fixed dose of rFSH which includes only 2,3-sialylation) with a multiple pregnancy rate at the same level as observed with oral agents and approximately half the rate reported with other gonadotropins (including other forms of FSH), supporting use of the dosing regimen disclosed herein in clinical practice.

### Conclusion

The PITS study reported herein is the first one using follitropin delta for stimulation in an intrauterine insemination protocol, and confirms the efficacy and benefits of the personalized dosing regimen of follitropin delta described herein.

### Addendum to the Example

The PITS study results updated as of June 2023 are consistent with the results reported and discussed above.

Table 9 below shows updated cumulative pregnancy outcomes up to 3 cycles (106 patients):

| | Cumulative pregnancy rate up |
|---|---|
| | to 3 cycles (106 patients) |
| Positive pregnancy test | 47 (44.3%) |
| Clinical pregnancy | 43 (40.6%) |
| Ongoing pregnancy | 39 (36.8%) |
| Live birth | 35 (33.0%) |

Thus, the updated results for the PITS study also show a higher cumulative clinical pregnancy rate (40.6% compared to 37.7%, pre update) and cumulative live birth rate (33% compared to 33%, pre update) were obtained compared to the results for the three prior art randomized clinical trials described above, and that improvement was achieved with only three cycles of IUI instead of four. Additionally, the multiple pregnancy rate in the PITS study [14% (6/43), with no triplets] was less than half that observed in the AMIGOS trial (31.8%), with a lower cancelation rate than that observed in the Dankert's trial. These results support the safety of the dosing regimen disclosed herein. While not wanting to be bound by theory, the use of a novel, human-derived recombinant FSH (follitropin delta), which has been shown to produce a milder ovarian stimulation in polycystic ovaries (PCO) patients, may be partly responsible for the reduced multiple pregnancy rate.

The PITS study showed that a personalised approach using individualized dosing of follitropin delta results in a higher clinical pregnancy rate (than, e.g., with use of, e.g., a fixed dose of rFSH which includes only 2,3-sialylation) with a multiple pregnancy rate at the same level as observed with oral agents and approximately half the rate reported with other gonadotropins (including other forms of FSH), supporting use of the dosing regimen disclosed herein in clinical practice.

### Additional Breakdown of Data

The Table 6 data have been broken down by patient AMH group as follows:

| | AMH (pmol/L) | | | |
|---|---|---|---|---|
| | <15.0 | 15.0-22.9 | ≥23.0 | |
| | (n = 60) | (n = 20) | (n = 22) | P value |
| Cumulative | 27 (45.0%) | 8 (40.0%) | 8 (36.4%) | 0.641 |
| clinical | | | | |
| pregnancy | | | | |
| Cumulative | 21 (35.0%) | 6 (30.0%) | 7 (31.8%) | 0.906 |
| live birth | | | | |

The breakdown demonstrates the improved cumulative clinical pregnancy rate (over the AMIGOS prior art values of 35,5%, 28,3% and 22.4%) for patients of AMH <15 pmol/L (45%), and patients with AMH ≥15 pmol/L (using data for patients with AMH 15.0 to 22.9 and those with AMH ≥23 pmol/L). Further, the breakdown demonstrates the improved cumulative clinical pregnancy rate (over the AMIGOS prior art values of 35,5%, 28,3% and 22.4%) for patients with PCOS (AMH ≥23.0 pmol/L) (36.4%).

Further, the breakdown demonstrates improved cumulative live birth rate (over the AMIGOS prior art values of 32,2%, 23,3% and 18.7%) for AMH patients for patients of AMH <15 pmol/L (35%), and patients with AMH ≥15 pmol/L (using data for patients with AMH 15.0 to 22.9 and those with AMH ≥23 pmol/L). Finally, the breakdown demonstrates the improved cumulative live birth rate (over the AMIGOS prior art values of 32,2%, 23,3% and 18.7%) for patients with PCOS (AMH ≥23.0 pmol/L) (31.8%). There is some similarity with the 32.2% results in the high AMH and PCOS patients but when the multiple pregnancy rate of the present trial (half that for the AMIGOS trial) is considered, the improvement is clear.

There have been disclosed hereinbefore the methods defined by the following numbered paragraphs:
1. A method for the treatment of infertility by intrauterine insemination in a patient having a serum AMH level of <15 pmol/L, comprising administering to the patient a composition comprising recombinant follicle stimulating hormone (rFSH) at a dose of from 0.048 μg to 0.052 μg rFSH per kg bodyweight of the patient per day.
2. The method according to paragraph 1, wherein the composition is administered at a dose of 0.05 μg rFSH per kg bodyweight of the patient per day.
3. The method according to paragraph 1, wherein the rFSH includes α2,6-sialylation and α2,3-sialylation.
4. The method according to paragraph 3, wherein 5% to 20% of the total sialylation of the rFSH is α2,6-sialylation and 80% to 95% of the total sialylation of the rFSH is α2,3-sialylation.
5. The method according to paragraph 1, wherein the rFSH has been produced or expressed in a human cell line.
6. The method according to paragraph 1, further comprising, prior to the administering, determining the serum AMH level of the patient.
7. The method according to paragraph 3, wherein the method is effective to increase one or both of pregnancy rate and live birth rate as compared to a method using rFSH that includes only is α2,3-sialylation.
8. The method according to paragraph 1, wherein the patient is of age <35 years.
9. The method according to paragraph 1, wherein the patient is of age 35 to 40 years.
10. The method according to paragraph 1, wherein the patient is of age >40 years.
11. The method according to paragraph 1, wherein the treatment of infertility by intrauterine insemination is controlled ovarian stimulation for intrauterine insemination.
12. The method according to paragraph 1, further comprising triggering ovulation in the patient by administering hCG.
13. The method according to paragraph 11, further comprising a step of intrauterine insemination.
14. A method for the treatment of infertility by intrauterine insemination in a patient having a serum AMH level of ≥15 pmol/L, comprising administering to the patient a composition comprising recombinant follicle stimulating hormone (rFSH) at a dose of from 0.025 μg to 0.0475 μg rFSH per kg bodyweight of the patient per day.
15. The method according to paragraph 14, wherein the patient has polycystic ovary syndrome (PCOS).
16. The method according to paragraph 14, further comprising, prior to the administering, determining the serum AMH level of the patient.
17. The method according to paragraph 14, wherein the rFSH includes α2,6-sialylation and α2,3-sialylation.
18. The method according to paragraph 17, wherein 5% to 20% of the total sialylation of the rFSH is α2,6-sialylation and 80% to 95% of the total sialylation of the rFSH is α2,3-sialylation.
19. The method according to paragraph 14, wherein the rFSH has been produced or expressed in a human cell line.
20. The method according to paragraph 16, wherein the method is effective to increase one or both of pregnancy rate and live birth rate as compared to a method using rFSH that includes only is α2,3-sialylation.
21. The method according to paragraph 14, wherein the patient is of age <35 years.
22. The method according to paragraph 14, wherein the patient is of age 35 to 40 years.
23. The method according to paragraph 14, wherein the patient is of age >40 years.
24. The method according to paragraph 14, wherein the treatment of infertility by intrauterine insemination is controlled ovarian stimulation for intrauterine insemination.
25. The method according to paragraph 14, further comprising triggering ovulation in the patient by administering hCG.
26. The method according to paragraph 25, further comprising a step of intrauterine insemination.

The invention claimed is:

1-22. (canceled)

23. A method of treating infertility by intrauterine insemination, comprising administering to a patient in need thereof a composition comprising recombinant follicle stimulating hormone (rFSH), wherein the patient has a serum Anti-Mullerian hormone (AMH) level of <15 pmol/L, and wherein the composition is administered at a dose of, or equivalent to, from 0.048 μg to 0.052 μg, rFSH per kg bodyweight of the patient per day.

24. The method according to claim 23, wherein the composition is administered at a dose of 0.05 μg rFSH per kg bodyweight of the patient per day.

25. The method according to claim 23, wherein the method further comprises, prior to the administering, determining the serum AMH level of the patient, and administering the dose to a patient having a serum AMH level of <15 pmol/L.

26. The method according to claim 23, wherein the patient is of age <35 years.

27. The method according to claim 23, wherein the patient is of age 35 to 40 years.

28. The method according to claim 23, wherein the patient is of age >40 years.

29. The method according to claim 23, wherein the rFSH includes α2,3- and α2,6-sialylation.

30. The method according to claim 23, wherein the rFSH includes α2,3- and α2,6-sialylation, wherein 5% to 20% of the total sialylation is α2,6-sialylation and 80% to 95% of the total sialylation is α2,3-sialylation.

31. The method according to claim 23, wherein the rFSH has been produced or expressed in a human cell line.

32. The method according to claim 23, wherein the method comprises controlled ovarian stimulation for intrauterine insemination.

33. The method according to claim 23, wherein the method further comprises a step of triggering ovulation and, optionally, a step of intrauterine insemination.

34. A method of treating infertility by intrauterine insemination, comprising administering to a patient in need thereof a composition comprising recombinant follicle stimulating hormone (rFSH), wherein the patient has a serum Anti-Mullerian hormone (AMH) level of ≥15 pmol/L, and wherein the composition is administered at a dose of, or equivalent to, from 0.025 μg to 0.0475 μg rFSH per kg bodyweight of the patient per day.

35. The method according to claim 34, wherein the patient has polycystic ovary syndrome (PCOS).

36. The method according to claim 34, wherein the composition is administered at a dose of 0.025 μg to 0.0475 μg rFSH per kg bodyweight of the patient per day.

37. The method according to claim 34, wherein the method further comprises, prior to the administering, determining the serum AMH level of the patient, and administering the dose to a patient having a serum AMH level of ≥15 pmol/L.

38. The method according to claim 34, wherein the rFSH includes α2,3- and α2,6-sialylation.

39. The method according to claim 34, wherein the rFSH includes α2,3- and α2,6-sialylation, wherein 5% to 20% of the total sialylation is α2,6-sialylation and 80% to 95% of the total sialylation is α2,3-sialylation.

40. A method of treating infertility by intrauterine insemination in a patient with PCOS, comprising administering to a PCOS patient in need thereof a composition comprising recombinant follicle stimulating hormone (rFSH), wherein the PCOS patient has a serum Anti-Mullerian hormone (AMH) level of ≥23 pmol/L, and wherein the composition is administered at a dose of, or equivalent to, from 0.025 μg to 0.0475 μg rFSH per kg bodyweight of the patient per day.

41. A method of treating infertility by intrauterine insemination in a patient with PCOS, comprising administering to a PCOS patient in need thereof a composition comprising recombinant follicle stimulating hormone (rFSH) that includes α2,3- and α2,6 sialylation, wherein the composition is administered at a daily dose of 3 to 7 μg rFSH.

42. The method of claim 41, wherein the patient has BMI of 30 kg/m² or more.
